(19) United States
(12) Patent Application Publication
(10) Pub. No.: US 2024/0225517 A1
(43) Pub. Date: Jul. 11, 2024

(54) METHOD FOR REMOTE PHYSIOLOGICAL GROUP BRAINWAVE SYNCHRONIZATION

(71) Applicant: SENS.AI INC., WHISTLER (CA)

(72) Inventors: Paola Telfer, Whistler (CA); Corey Julihn, Langley (CA)

(21) Appl. No.: 18/559,319

(22) PCT Filed: May 6, 2022
(86) PCT No.: PCT/IB2022/054238

§ 371 Date: Nov. 6, 2023

Related U.S. Application Data
(60) Provisional application No. 63/185,245, filed on May 6, 2021.

(51) Int. Cl. A61B 5/375; A61B 5/00

(52) U.S. Cl. CPC A61B 5/375; A61B 5/6803

(57) ABSTRACT

Methods for local and remote physiological group brainwave synchronization are provided. The inventive methods rely on electroencephalography (EEG) devices for measuring the electrical activity of the brain. Said biological signals are processed to determine the level of phase synchronization within the group and individuals are rewarded using biofeedback techniques including audio and visual stimulus. The inventive methods provide for (1) brainwave group phase synchronization, (2) remote and large group synchrony and (3) variations that allow for applications ranging from flow, engagement, affinity, empathy, social closeness, creativity, brainstorming, communication, reconciliation, and trauma release.

### PRIOR RELATED APPLICATIONS

This application claims the benefit of priority of prior-filed U.S. Provisional Application 63/185,245, filed May 6, 2021.

### FIELD OF THE INVENTION

The present invention relates to methods for physiological group brainwave synchronization. Specifically, the invention pertains to using electroencephalography (EEG) and electrocardiogram (EKG) and other biological sensors for measuring and altering the electrical activity of the brains of multiple individuals in coordination at the same time. In particular, in this coordinated approach, the phase of selected brainwave frequencies for individuals are shifted to be in closer phase synchronization with other members of the group. This technique can be used to modulate or improve overall group states including engagement, affinity, empathy, social closeness, flow, creativity, brainstorming, communication, reconciliation, and trauma release.

### BACKGROUND OF THE INVENTION

Historically, methods for synchronization of biological signals across a group of individuals have been rooted in meditation practices. More recently technical approaches have been developed based on the synchronization of heart rate signals. These methods utilize electrocardiogram (ECG or EKG) sensors to measure the electric fields of the heart and provide feedback to the group using audio and visual cues. To date, heart rate group synchronization has been deployed at festivals, conferences and in retreat settings, in most cases using custom, one-off equipment that is manually operated.

Signal processing techniques can be used to determine the frequencies present in a signal, along with their components, including amplitude and phase. Phase synchronization measures the timing of two or more signals at a given frequency and provides a measure of how in-time or in-sync those signals are. Applying these measures to brainwaves, one can measure the electrical activity of the brain using Electroencephalogram (EEG) sensors and then determine the level of phase synchronization over time at different locations of a single brain. Furthermore, one can analyze the level of phase synchronization over time across multiple individuals' brains. The level of phase synchronization is a scale from 0 to 1 or 0% to 100%.

Recent research has begun to analyze the performance of individuals in a group compared to the level of brainwave synchronization across individuals in the group. One such study discussed in the paper Brain-to-Brain Synchrony Tracks Real-World Dynamic Group Interactions in the Classroom, Dikker et al., 2017, found that “synchronized neural activity across a group of students predicts (and possibly underpins) classroom engagement and social dynamics” such as group affinity, empathy, and social closeness.

Influencing biometric signals from the body crosses many disciplines and methods including medicine, therapy, meditation, breathing exercises, biofeedback, neurofeedback and biostimulation. Neurostimulation is one form of biostimulation which involves the purposeful modulation of nervous system activity. One such method of neurostimulation known as Photobiomodulation (PBM) uses modulating near-infrared light to stimulate the nervous system.

Photobiomodulation is a form of infrared light therapy. Infrared light therapy can have positive effects on the skin, metabolic processes, the nervous system and immune system. It has been shown to increase collagen production for healthier skin.

Photobiomodulation techniques can stimulate the mitochondria in cells through the transfer of energy. Inside mitochondria, cytochrome oxidase has the ability to absorb red and near infrared light and convert it into energy—adenosine triphosphate (ADT). Transcranial photobiomodulation systems often transmit light at a wavelength between 633 and 810 nanometers with 810 nm being an ideal wavelength due to its ability to penetrate further into biological tissue.

Further, transcranial photobiomodulation is a neurotechnology technique used to modulate or alter an individual's brain activity creating a perceptible change in mental state which can be seen through changes in the electrical activity of the brain. Brainwave states can be defined as the collective electrical activity of a brain over a period of time; which can then be classified into a mental state such as tired, focused, stressed, creative, etc.

The inventors are not aware of any other published methods for creating states of group brainwave synchronization through biofeedback or biostimulation. Furthermore, the inventors are not aware of publications suggesting that group brainwave synchronization can be achieved through biofeedback or biostimulation. Accordingly, the inventors provide the current invention as a novel method for influencing brain signals across a group of individuals in order to increase group brainwave synchronization. Using this method on a group of individuals results in improved group states including engagement, affinity, empathy, social closeness, flow, creativity, brainstorming, communication, reconciliation, and trauma release.

### SUMMARY OF THE INVENTION

The present invention relates to methods for physiological group brainwave synchronization, wherein each individual in the group has their biological signals measured in real-time using biometric sensors including 1 or more Electroencephalogram (EEG) sensors. Said biological signals are processed to determine the level of phase synchronization with the group and individuals in the group are rewarded or stimulated using biofeedback techniques including audio and visual stimulus. The biofeedback loop continues shifting the phase of individuals' brainwaves toward a common phase synchronization. Heart synchronization is also achievable using the methods and apparatus of the present invention and embodiments are provided accordingly.

The present invention utilizes EEG recorded from locations including at Fz, Cz, and Pz according to the international 10-20 placement system. In other embodiments additional or alternative EEG electrode placements may be utilized. Accordingly, the present invention can be used for group brain synchronization of brainwave bands including delta, theta, alpha, alpha-theta, low beta, mid meta, high beta, and gamma waves. This method may be applied to induce group flow states that would be performance enhancing for teams; this includes both physical and mental performance. Another application of this method is to enhance emotional opening and reflective group states. In yet another application this method can be used to improve creative brainstorming for teams.

The inventive method relies on reading biosignals which change due to changes in the body's electric field and are amplified and converted into a digital signal and sent to a computer, phone, wearable, server and/or other device through wired or wireless connection such as Bluetooth, WiFI, cellular, or internet where is may be processed, stored, displayed, and/or interpreted.

Furthermore, timing of biological signals is an important factor in the present invention. Therefore the present method benefits if the timing of said biological signals is synchronized to the master device accounting for delays in electronics and transmission including wireless signal transmission. Further, the total time from reading a biological signal to the time a reward stimulus is received should be less than 500 ms. In a local embodiment, all participants in the group are in the same general physical area. With relatively low transmission delays the system may directly provide the feedback level to each individual's feedback device.

### BRIEF DESCRIPTION OF THE DRAWINGS

FIG. 1 shows a wearable headset and connected mobile device and computer system in accordance with an embodiment of the present invention

FIG. 2 illustrates 3 signals with varying amplitude and similar frequencies where signal A and B share the same phase and signal C is out of phase with signal A and B.

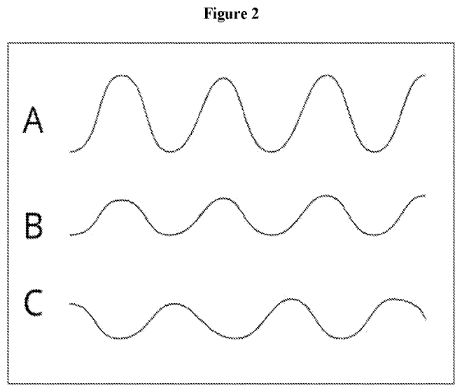

### DETAILED DESCRIPTION OF THE INVENTION

Each of the examples of the embodiments of the invention are provided according to an explanation of the specifics of the invention, and should not be considered as a limitation of the invention. To those skilled in the art, it is understood that modifications can be made in the present invention within the scope or spirit of the apparatus, system, and methods of the present invention.

Further, in this invention, the terms such as “person”, and “user”, and “wearer”, and “patient”, and “human”, and “individual” and “subject” are used interchangeably to refer to a person using the invention. “Treatment” or “stimulation” or “therapy” or “training” or “session” as used herein, covers the use of the invention by one or more persons to obtain benefits or intended results in the person/user/wearer/patient/human/individual, aimed at synchronization of biological signals.

Turning now to the inventive embodiments, and with specific reference to the accompanying drawings, the invention is described in greater detail.

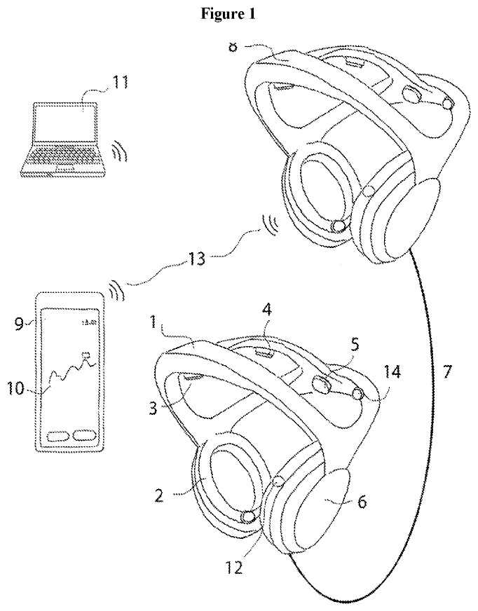

In one aspect, the present invention provides EEG and PPG sensors in a head mounted device 1 and 8 with headphones 2 and 6 illustrated in FIG. 1. In an embodiment illustrated in FIG. 1, the headphones of the present invention combine EEG (electroencephalography) sensors 3, 4 and 5 for EEG measurement and phoplethysmography (PPG) sensor 12 for heart rate and heart rate variability (HRV) measurement in a wearable head mounted device with headphones. In an embodiment, a PPG sensor 12 is incorporated inside an over-ear headphone design which reduces ambient noise allowing for increased accuracy. The present invention provides a wearable head mounted headphone set 1 and 8 with embedded biometric sensors that collect physiological signals from the user. The device includes Bluetooth (wireless) audio and data transmission 13 which may be used to connect the device 1 and 2 to control unit which may be a smartphone or mobile device 9, with graphic touchscreen display 10, and said control unit 9 has wireless wi-fi connection with remotely located master control unit which may be a computer 11. The device 1 and 8 may also include a rechargeable battery, speakers, microphone. The device 1 and 8 may further include a detachable wire 7 allowing multiple devices to be connected together. In one embodiment, the present invention includes one or more Photobiomodulation (PBM) LEDs 14 embedded in the head mounted device 1 and 8.

In another embodiment, said control unit and wearable device may be combined into a single wearable device.

In yet another embodiment, said graphic touchscreen display on the control unit may be a virtual reality (VR), augmented reality (AR), or mixed reality (MR) display.

The present invention may be applied to groups of 2 or more people wherein each person in the group wears said head mounted device with biometric sensors. In the preferred embodiment each member of the group also utilizes a mobile device which controls their wearable devices and collects the biometric signal data through a wireless connection. In an alternative embodiment the control units may be wired to the wearable device.

When beginning a group synchronization session one member of the group will be designated as the host. The host will use their control unit to initiate the group session and will invite other participants to join the group. All participants will connect their control units to their wearable devices by establishing a wireless connection.

The present invention requires sensor timing synchronization that is accurate enough to support phase synchronization calculations across participants, devices, and sensors. This accuracy requirement is subject to the frequencies that are selected for synchronization. For example, a 10 Hz signal has a period of 100 milliseconds. Taking two 10 Hz sine waves that are perfectly in phase and shifting one by 50 milliseconds results in the two waves being completely out of phase. A timing accuracy of plus or minus 5 milliseconds could result in the signals being out of phase by 10 milliseconds, and result in up to 15% loss of accuracy when calculating phase synchronization. Therefore the present invention includes techniques for synchronizing the timing across participants, devices, and sensors that is accurate to within 1 millisecond.

In one embodiment, the host control unit will send a command to the host device wherein upon receiving the command, the host device marks the time as time zero. Furthermore the host device when transmitting biological signal data to the host control unit includes timestamp relative to time zero. The present invention may indicate to the host that the device has established time zero by displaying a message on the control unit and with an indicator light on the device itself.

Once the host device has established time zero, all other wearable devices in the group must be synchronized to the identical time zero mark. In one embodiment of the present invention, a device that has time zero may be plugged into a device without time zero. Any device with time zero will periodically transmit data indicating the duration of time since time zero. A device without time zero will listen for the data transmission in order to mark time zero.

Those skilled in the art will understand that different data protocols or techniques may be used in order to synchronize the timing of all signal data across the group. In one embodiment, data synchronization could be established using wireless communication including but not limited to infrared communication, near-field communication, WiFi, or bluetooth. Wherein each communication method could utilize one or more techniques in order to establish common timing across sensors and wearable devices.

After each device has established the same time zero, the devices begin transmitting biological signal data to their respective control units including a timestamp relative to time zero. Wherein said biological signal data includes EEG signal data from one or more locations on each person's brain. In one embodiment, this signal data is processed by each control unit. Signal processing may include various techniques known to those skilled in the art, including noise filters (i.e. lowpass, highpass, etc.) and analysis techniques (i.e. Fourier transform, Wavelet analysis, etc.). Each control unit further filters the signals into narrowband signals for each combination target of sensor locations and synchronization frequencies. For example, if the group is attempting to synchronize 10 Hz at PZ, a 1 Hz wide signal centered at 10 Hz may be used for the narrowband signal. Each control unit then calculates the phase angles of each narrowband signal. Next each control unit transmits processed data including the signal phase angles and the duration of time since time zero.

In another embodiment, each control unit may relay the raw or partially processed signal data to a master control unit. In this embodiment the master control unit may be located near the group or it may be a remotely located server. In this embodiment the master control unit may execute some or all of the signal processing. In another embodiment, one of the participant's control unit, may serve as the master control unit for the group.

Upon receiving the signal data including phase angles from each control unit the master control unit in the present invention shall determine a target phase timing for the group. Various calculations may be used to determine the target phase, with some methods being suited to smaller group sizes and other methods working for larger groups.

In one embodiment, the master control unit determines the target phase for 1 or more target brainwave frequency for the group, and said target phase is adapted over time to optimize the overall level of synchronization wherein:
1. The initial target phase is derived from the median phase of the entire group.
2. The master control unit selects a predetermined percent of the individuals in the group that are closest to the target phase and re-calculates the target phase using this subgroup, a minimum group size may be used.
3. The target phase relative to time zero is returned to each control unit.
4. The master control unit periodically calculates a new target phase for the group attempting to maximize phase synchronization of the group.
   a. The master control unit calculates the phase width from step 2; where phase width is the maximum level of phase synchronization for an individual in the subgroup compared to the target phase. The master control unit may utilize a minimum and maximum phase width.
   b. Next the master control unit adapts the target phase overtime, by shifting the target phase in order to maximize the number of individuals within the phase width.
   c. Periodically, the master control unit may return to step 2.
5. The target phase relative to time zero is returned to each control unit.

In this embodiment, changes to the target phase may be limited to a maximum rate of change. Further, the timing of re-calculating the target phase may be predetermined, configured by the group, or adjusted over time by the master control unit.

In another embodiment of the present invention, the master control unit uses a mean or median calculation to determine the target phase of the group.

In yet another embodiment, the master control unit may use additional signal metrics when determining which individuals are included in the target phase calculation. In one embodiment, individuals in the group are only included in the target phase calculation if they reach a minimum average amplitude level for the target frequency.

In still another embodiment, each individual control unit utilizes a predetermined target phase relative to time zero. In this embodiment, the master control unit is not required and control units are not required to communicate with one another.

In another embodiment one or more individuals in the group may be classified by the system as high priority. These participants may be selected for any reason. Some possible examples of a high priority participant include a leader, teacher, mentor, or an expert. In one version of this embodiment the high priority individual(s) are used to set the target phase. In another version of this embodiment the high priority individual(s) are given higher weighting when calculating the target phase.

In another embodiment, all individuals in the group receive a weighting based on one or more biometric indicators including: heart rate variability (HRV), heart rate (HR), heart coherence, EEG band power, EEG band amplitude, EEG band phase synchronization. Wherein the target phase calculation is weighted according to each individual's weighting.

The control units and wearable devices of the present invention utilize the target phase for the group and biofeedback techniques in order to influence individuals in the group to shift the phase of their biometric signals toward the target phase. Wherein a control unit may calculate the percentage of phase synchronization an individual has with the target phase and provide audio, visual or other stimuli as rewards. In one embodiment, the control unit may also provide biofeedback to individuals based on 1 or more of the follow calculations:
1. An individual's overall frequency power or amplitude based on the targeted frequency or frequencies.
2. The entire group's level of phase synchronization for 1 or more target brainwave frequencies.
3. The level of phase synchronization for a subset of the group made up of individuals within a given percentage of the target phase 1 or more target brainwave frequency.
4. An individual's level of phase synchronization for 1 or more target brainwave frequency compared to the target phase of the group.
5. Other biometrics including but not limited to: heart rate variability (HRV), heart coherence, individual brain synchronization, and breathing rate.

In a remote embodiment, participants may be in physically disparate locations, and rely on a central server as the master control unit device. In this embodiment additional data timing synchronization methods must be utilized. Where known methods such as Network Time Synchronization (NTP) can provide or Precision Time Protocol (PTP) can be used to address latency between the master control unit and the distributed control units of the group. The master control unit can then establish a real-world time as time zero. Control units still need to be synchronized to the wearable devices and sensors. In one such embodiment control units may be directly connected via a wire to the wearable device. In another such embodiment, the control unit may temporarily plug into the wearable device to transmit the duration of time since time zero. Still yet another embodiment, the control unit and device may implement a wireless time synchronization protocol. Where said protocol is implemented over Bluetooth, there is currently no standard time synchronization protocol. However there are various techniques such as (Asgarian & Najafi 2001) which demonstrate sub-millisecond precision.

In yet another embodiment, the method includes techniques for biostimulation or neurostimulation. Said stimulation techniques may include electrical stimulation, ultrasound, pulse electric magnetic field (PEMF) or light stimulation (Photobiomodulation). Separately or in combination with biofeedback, the system may use stimulation to shift the phase of 1 or more targeted brainwave frequencies of an individual toward the target phase.

In one embodiment, the wearable device includes one or more Photobiomodulation (PBM) LEDs. Wherein the device may utilize photobiomodulation to provide additional energy to each individual's brains. Photobiomodulation techniques may be applied prior to a group synchronization session, during the session, or after the session.

In one embodiment, heart biometric signals may be used to induce group synchrony through biofeedback, HRV training and breathing techniques. Wherein the control unit processes said biological signal data including determining the phase of the heart rate for individuals within the group. This embodiment may be implemented using the remote timing synchronization techniques of the present invention. Prior art attempts have been made to synchronize group heart rate signals. These prior art attempts rely on a single device connecting all sensors, and all participants remaining in the same physical location—i.e., not situated in remote locations from each other. The present invention allows for multiple devices, and for participants to be remotely located, or to move to remote locations during or prior to starting a group synchronization feedback session.

In one embodiment, the present method may utilize a single device which incorporates all sensors, signal processing, and feedback techniques. In another embodiment, each individual in the group has a device which includes sensors and feedback mechanisms, wherein each device transmits sensor data to a master device where the group signals are processed and results returned to the individual devices. In yet another embodiment, the master device is a remote server, and the individuals wearing their own devices may be in different physical locations from the master server.

One of skill in the art will realize that variations on the embodiments of the invention provided herein are possible without departing from the scope and spirit of the disclosure. Solely by way of example, the skilled artisan will understand that alternate placements of sensors and LED's in the disclosed headset and methods are possible while still falling within the scope of the claimed invention.

What is claimed is:

1. A method for physiological group brainwave synchronization comprising:
measuring biological signals of at least two individuals using biometric sensors;
processing the measured biological signals to determine a level of phase synchronization of the biological signals of the at least two individuals;
providing biofeedback stimuli to the at least two individuals to induce a shift in the phase of the biological signals of the at least two individuals; and
establishing a biofeedback loop to shift the phase of the biological signals of the at least two individuals toward a target phase synchronization of the biological signals of the at least two individuals.

2. The method of claim 1, wherein the biometric sensors are EEG sensors.

3. The method of claim 2, wherein the EEG sensors are placed in locations where they are able to read the electrical field of the brain.

4. The method of claim 1, wherein the biometric sensors are one or more of EEG sensors, PPG sensors and EKG sensors.

5. The method of claim 4, wherein the PPG sensors or EKG sensors are configured to measure heart rate and heart rate variability and the EEG sensors are placed in locations where they are able to read the electrical field of the brain.

6. The method of claim 1, wherein the biofeedback stimuli may be provided in the form of an audio stimulus or a visual stimulus or tactile feedback or a combination thereof.

7. The method of claim 6, wherein the biofeedback stimuli may be provided in the form of one or more of a PBM LED and a VR, AR or MR display.

8. The method of any of the preceding claims wherein the at least two individuals are fitted with a head mounted device incorporating the biometric sensors, one or more modalities for providing the biofeedback stimuli, a control unit and wireless transmission means for connecting each head mounted device with a master controller and one or more mobile devices.

9. The method of claim 8 further comprising the step of establishing time zero for each head mounted device fitted to the at least two individuals, wherein the measured biological signals are temporally synchronized.

10. The method of claim 8, wherein the control unit is programmed with a predetermined target phase synchronization.

11. The method of claim 8, wherein the target phase synchronization is based on the biological signals of a leader.

12. The method of claim 8, wherein the target phase synchronization is based on real time measurement of the biological signals of the at least two individuals.

13. The method of any of claims 8 through 12, wherein the control unit of each head mounted device provides biometric indicators of each of the at least two individuals to the master controller, and wherein the master controller uses a weighting to calculate the target phase synchronization, and wherein the master controller provides instructions to the control unit of each head mounted device for the target phase synchronization for each of the at least two individuals.

14. The method of any of claims 8 through 13, wherein the at least two individuals are positioned in locations remote from each other.

15. A method for physiological group heart synchronization comprising:
measuring biological signals of at least two individuals using biometric sensors capable of measuring heart electrical activity;
processing the measured biological signals to determine a level of phase synchronization of the biological signals of the at least two individuals;
providing biofeedback stimuli to the at least two individuals to induce a shift in the phase of the biological signals of the at least two individuals; and
establishing a biofeedback loop to shift the phase of the biological signals of the at least two individuals toward a target phase synchronization of the biological signals of the at least two individuals;
wherein the at least two individuals are fitted with a head mounted device incorporating the biometric sensors, one or more modalities for providing the biofeedback stimuli, a control unit and wireless transmission means for connecting each head mounted device with a master controller and one or more mobile devices.

16. The method of claim 15, wherein the biometric sensors are PPG sensors or EKG sensors or a combination thereof.

17. The method of claim 16, wherein the at least two individuals are positioned in locations remote from each other.
